(19) United States
(12) Patent Application Publication
(10) Pub. No.: US 2023/0089692 A1
(43) Pub. Date: Mar. 23, 2023

(54) DYNAMIC COVALENT POLYMERIZATIONS WITH ELEMENTAL SULFUR AND SULFUR PREPOLYMERS

(71) Applicant: ARIZONA BOARD OF REGENTS ON BEHALF OF THE UNIVERSITY OF ARIZONA, Tucson, AZ (US)

(72) Inventors: Dong-Chul Pyun, Tucson, AZ (US); Yueyan Zhang, Tucson, AZ (US)

(21) Appl. No.: 18/051,342

(22) Filed: Oct. 31, 2022

Related U.S. Application Data
(63) Continuation-in-part of application No. 16/322,646, filed on Feb. 1, 2019, now abandoned.
Application No. 16/322,646 is a continuation-in-part of application No. 17/156,238, filed on Jan. 22, 2021.
Application No. 17/156,238 is a continuation-in-part of application No. PCT/US2019/054368, filed on Oct. 2, 2019.
Application No. 17/156,238 is a continuation-in-part of application No. 15/839,344, filed on Dec. 12, 2017, now abandoned.
Application No. 15/839,344 is a continuation-in-part of application No. PCT/US16/42057, filed on Jul. 13, 2016.
Application No. 17/156,238 is a continuation-in-part of application No. 15/857,273, filed on Dec. 28, 2017, now Pat. No. 10,920,020.
Application No. 15/857,273 is a continuation-in-part of application No. PCT/US16/42057, filed on Jul. 13, 2016.
Application No. 15/857,273 is a continuation-in-part of application No. 15/287,118, filed on Oct. 6, 2016, now Pat. No. 10,833,330.
Application No. 15/287,118 is a continuation of application No. 14/622,429, filed on Feb. 13, 2015, now Pat. No. 9,567,439.
Application No. 14/622,429 is a continuation-in-part of application No. 14/237,659, filed on Mar. 11, 2014, now Pat. No. 9,306,218.
(60) Provisional application No. 62/369,382, filed on Aug. 1, 2016.
(60) Provisional application No. 62/740,372, filed on Oct. 2, 2018.
(60) Provisional application No. 62/433,050, filed on Dec. 12, 2016.
(60) Provisional application No. 62/440,576, filed on Dec. 30, 2016.
(60) Provisional application No. 62/191,760, filed on Jul. 13, 2015.
(60) Provisional application No. 62/203,525, filed on Aug. 11, 2015.
(60) Provisional application No. 62/210,170, filed on Aug. 26, 2015.
(60) Provisional application No. 62/212,188, filed on Aug. 31, 2015.
(60) Provisional application No. 62/306,865, filed on Mar. 11, 2016.
(60) Provisional application No. 62/313,010, filed on Mar. 24, 2016.
(60) Provisional application No. 62/329,402, filed on Apr. 29, 2016.
(60) Provisional application No. 62/039,561, filed on Aug. 20, 2014.
(60) Provisional application No. 62/039,588, filed on Aug. 20, 2014.
(60) Provisional application No. 62/017,750, filed on Jun. 26, 2014.
(60) Provisional application No. 61/940,102, filed on Feb. 14, 2014.
(60) Provisional application No. 61/685,847, filed on Mar. 26, 2012.
(60) Provisional application No. 61/574,957, filed on Aug. 12, 2011.
(60) Provisional application No. 61/574,903, filed on Aug. 11, 2011.

(51) Int. Cl. C08G 75/16; C08L 81/04; C01B 17/96; A61K 31/795; H01M 4/60; A01N 59/02; A01N 41/12; C01B 3/04; C04B 35/63; C04B 35/64; H01M 4/04; H01M 4/58; H01M 4/66

(52) U.S. Cl. CPC C08G 75/16; C08L 81/04; C01B 17/96; A61K 31/795; H01M 4/606; A01N 59/02; A01N 41/12; C01B 3/042; C04B 35/6303; C04B 35/64; H01M 4/0404; H01M 4/5815; H01M 4/608; H01M 4/664; H01M 10/052

(57) ABSTRACT

An iterative approach to dynamic covalent polymerizations of elemental sulfur with functional comonomers to prepare sulfur prepolymers that can further react with other conventional, commercially available comonomers to prepare a wider class of functional sulfur polymers. This iterative method improves handling, miscibility and solubility of the elemental sulfur, and further enables tuning of the sulfur polymer composition. The sulfur polymers may be a thermoplastic or a thermoset for use in elastomers, resins, lubricants, coatings, antioxidants, cathode materials for electrochemical cells, and polymeric articles such as polymeric films and free-standing substrates.

### CROSS-REFERENCES TO RELATED APPLICATIONS

This application is a continuation-in-part and claims benefit of U.S. patent application Ser. No. 16 322,646 filed Feb. 1, 2019, which is a 371 of PCT Patent Application No. PCT/US2017/041929 filed Jul. 13, 2017, which claims benefit of U.S. Provisional Application No. 62/369,382 filed Aug. 1, 2016, the specification(s) of which is/are incorporated herein in their entirety by reference.

This application is a continuation-in-part and claims benefit of U.S. patent application Ser. No. 17/156,238 filed Jan. 22, 2021, which is a continuation-in-part and claims benefit of PCT/US2019/054368 filed Oct. 2, 2019, which claims benefit of U.S. Provisional Application No. 62/740,372, filed Oct. 2, 2018, the specification(s) of which is/are incorporated herein in their entirety by reference.

U.S. 17/156,238 is also a continuation-in-part and claims benefit of U.S. patent application Ser. No. 15/839,344 filed Dec. 12, 2017, which is a non-provisional and claims benefit of U.S. Provisional Patent Application No. 62/433,050 filed Dec. 12, 2016, the specification(s) of which is/are incorporated herein in their entirety by reference.

U.S. Ser. No. 15/839,344 is also a continuation-in-part and claims benefit of PCT Patent Application No. PCT/US2016/042057 filed Jul. 13, 2016, the specification(s) of which is/are incorporated herein in their entirety by reference.

U.S. Ser. No. 17/156,238 is also a continuation-in-part and claims benefit of U.S. patent application Ser. No. 15/857,273 filed Dec. 28, 2017, now U.S. Pat. No. 10,920,020, which is a non-provisional and claims benefit of U.S. Provisional Application No. 62/440,576 filed Dec. 30, 2016, the specification(s) of which is/are incorporated herein in their entirety by reference.

U.S. Ser. No. 15/857,273 is also a continuation-in-part and claims benefit of PCT Application No. PCT/US2016/042057 filed Jul. 13, 2016, the specification(s) of which is/are incorporated herein in their entirety by reference.

PCT Application No. PCT/US2016/042057 claims benefit of U.S. Provisional Application No. 62/191,760 filed Jul. 13, 2015, U.S. Provisional Application No. 62/203,525 filed Aug. 11, 2015, U.S. Provisional Application No. 62/210,170 filed Aug. 26, 2015, U.S. Provisional Application No. 62/212,188 filed Aug. 31, 2015, U.S. Provisional Application No. 62/306,865 filed Mar. 11, 2016, U.S. Provisional Application No. 62/313,010 filed Mar. 24, 2016, and U.S. Provisional Application No. 62/329,402 filed Apr. 29, 2016, the specification(s) of which is/are incorporated herein in their entirety by reference.

U.S. Ser. No. 15/857,273 is also a continuation-in-part and claims benefit of U.S. patent application Ser. No. 15/287,118 filed Oct. 6, 2016, now U.S. Pat. No. 10,833,330, which is a continuation-in-part and claims benefit of U.S. patent application Ser. No. 14/622,429 filed Feb. 13, 2015, now U.S. Pat. No. 9,567,439, which claims benefit of U.S. Provisional Application No. 62/039,561 filed Aug. 20, 2014, U.S. Provisional Application No. 62/039,588 filed Aug. 20, 2014, U.S. Provisional Application No. 62/017,750 filed Jun. 26, 2014, and U.S. Provisional Application No. 61/940,102 filed Feb. 14, 2014, the specification(s) of which is/are incorporated herein in their entirety by reference.

Furthermore, U.S. Ser. No. 14/622,429 is also a continuation-in-part and claims benefit of U.S. patent application Ser. No. 14/237,659 filed Mar. 11, 2014, now U.S. Pat. No. 9,306,218, which is a 371 of PCT Application No. PCT/US2012/050602 filed Aug. 13, 2012, which claims benefit of U.S. Provisional Application No. 61/685,847 filed Mar. 26, 2012, U.S. Provisional Application No. 61/574,957 filed Aug. 12, 2011, and U.S. Provisional Application No. 61/574,903 filed Aug. 11, 2011, the specification(s) of which is/are incorporated herein in their entirety by reference.

### STATEMENT REGARDING FEDERALLY SPONSORED RESEARCH OR DEVELOPMENT

This invention was made with government support under Grant No. 1305773 awarded by National Science Foundation. The government has certain rights in the invention.

### FIELD OF THE INVENTION

The present invention relates to polymeric compositions and materials prepared from elemental sulfur. The methods of forming sulfur polymers described herein can solubilize elemental sulfur, enhance rate of reactions and widen the scope of accessible comonomers that can copolymerize with elemental sulfur.

### BACKGROUND OF THE INVENTION

An incredible abundance of elemental sulfur, nearly 7-million tons, is generated as a waste byproduct from hydrodesulfurization of crude petroleum feedstocks, which converts alkanethiols and other (organo) sulfur compounds into S₈. Before the invention of the inverse vulcanization process, there were only a limited number of synthetic methods available to utilize and modify elemental sulfur. Current industrial utilization of elemental sulfur is centered around sulfuric acid, agrochemicals, and vulcanization of rubber. For example, elemental sulfur is used primarily for sulfuric acid and ammonium phosphate fertilizers, where the rest of the excess sulfur is stored as megaton-sized, above ground sulfur towers.

While sulfur feedstocks are plentiful, sulfur is difficult to process. In its original form, elemental sulfur consists of a cyclic molecule having the chemical formulation S8 Elemental sulfur is a brittle, intractable, crystalline solid having poor solid state mechanical properties, poor solution processing characteristics, and there is a limited slate of synthetic methodologies developed for it. Hence, there is a need for the production of new materials that offer significant environmental and public health benefits to mitigate the storage of excess sulfur in powder, or brick form.

Elemental sulfur has limited solubility and miscibility with the vast range of commercially available comonomers, such as vinylic and unsaturated comonomers that are widely used in the chemical industry. In fact, elemental sulfur is only compatible with a small scope of conventional, industrially-used comonomers. Hence, there remains a need for methods that can provide high sulfur content polymers prepared from elemental sulfur. The present invention overcomes this chemical incompatibility of elemental sulfur by using a facile two-stage process that makes accessible the wider range of commercial monomers.

### SUMMARY OF THE INVENTION

It is an objective of the present invention to provide for functional sulfur polymers having a high molecular weight and sulfur content, and methods of making said sulfur polymers. Embodiments of the invention are given in the dependent claims. Embodiments of the present invention can be freely combined with each other if they are not mutually exclusive.

The subject disclosure features the copolymerization of elemental sulfur with comonomers, such as vinylic and other unsaturated comonomers, to prepare more soluble and more processable sulfur prepolymers that can be further reacted with other conventional, commercially available comonomers to prepare a wider class of functional sulfur copolymers. This approach is advantageous for enabling the incorporation of readily available comonomers into sulfur containing copolymers. An iterative approach is required since elemental sulfur is only compatible with a small scope of commercially available comonomers. In one embodiment, this iterative approach involves a two step process in which sulfur is melted at a temperature T>120° C. and reacted with vinylic comonomers (e.g., styrene) to form a sulfur prepolymer, and the sulfur prepolymer is directly melted or diluted in an organic solvent (e.g., 1,2-dichlorobenzene) and a second comonomer is added to form a target copolymer. Elevated temperatures are necessary for these copolymerizations to occur.

According to some embodiments, the present invention features an iterative method of synthesizing a sulfur polymer. The method may comprise providing elemental sulfur, heating the elemental sulfur to form a molten sulfur comprising dynamic sulfur-sulfur (S—S) bonds, adding one or more first comonomers to the molten sulfur, wherein each first comonomer comprises at least one reactive functional group, wherein the one or more first comonomers and molten sulfur copolymerize by a reaction of the sulfur radicals with the reactive functional groups of the one or more first comonomers to form a sulfur prepolymer, heating the sulfur prepolymer to form a molten sulfur prepolymer comprising the dynamic S—S bonds, wherein heating enables thermal activation and scission of the dynamic S—S bonds from said molten sulfur prepolymer to form sulfur-containing radical species, and adding one or more second comonomers to the molten sulfur prepolymer, wherein each of the one or more second comonomers comprises at least one reactive functional group, wherein the one or more second comonomers and the molten sulfur prepolymer copolymerize by a reaction of the sulfur-containing radical species with the reactive functional groups of the one or more second comonomers to form the sulfur polymer. This iterative method improves processability of the elemental sulfur in producing the desired sulfur polymer by initially copolymerizing the elemental sulfur with the one or more first comonomers to prepare the sulfur prepolymer that is tailored to be miscibly or solubly compatible with the one or more second comonomers.

One of the unique inventive technical features of the present invention is the iterative approach of reacting sulfur with a small amount of a commonly used comonomer (e.g., styrenics, allylics, (meth)acrylates, norbornenes) to prepare a sulfur prepolymer that is significantly more soluble and miscible with other conventional comonomers. The high content of dynamic S—S bonds in these sulfur prepolymers surprisingly allows for facile post-polymerization modifications and reactions of the sulfur prepolymers with the other Non-limiting examples of conventional comonomers include common alkynes and vinylic/unsaturated comonomers (e.g., functional styrenics, allylics, methacrylates, acrylates, norbornenes, acrylonitrile, vinylpyridines, maleimides, and isobutylenes). Furthermore, this iterative approach allows for the direct preparation of crosslinked polymer networks and the ability to increase the molecular weight of these sulfur polymers. None of the presently known prior references or work has the unique inventive technical feature of the present invention.

Another unique and inventive technical feature of the present invention is that the first comonomers and second comonomers are selected such that the sulfur prepolymer and the second comonomers are miscibility or solubly compatible with each other. Without wishing to limit the present invention to a particular theory or mechanism, this feature enables the reaction of elemental sulfur with a wide range of commercial monomers. By selecting specific first comonomers for the first polymerization reaction with elemental sulfur, the resulting sulfur prepolymer is miscibility or solubly compatible with the second comonomer, as compared to elemental sulfur. Furthermore, the second comonomer is selected to be miscibility or solubly compatible with the sulfur prepolymer, even if the second monomer is immiscible with elemental sulfur. Thus, the novel feature of the present invention allows for the polymerization of monomers that would otherwise be immiscible with liquid elemental sulfur. None of the presently known prior references or work has the unique inventive technical feature of the present invention.

Any feature or combination of features described herein are included within the scope of the invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE DRAWINGS

The features and advantages of the present invention will become apparent from a consideration of the following detailed description presented in connection with the accompanying drawings in which:

FIG. 1 shows a non-limiting example of an iterative reaction schematic of forming a poly(styrene-random-sulfur) prepolymer that further reacts with an unsaturated comonomer to form a functional sulfur polymer.

FIG. 2 shows another non-limiting reaction schematic of the poly(styrene-random-sulfur) prepolymer that reacts with n-butyl acrylate to form a poly(styrene-random-sulfur-n-butyl acrylate) terpolymer. FIG. 2 further shows a non-limiting example of a 1H NMR spectra of the poly(styrene-random-sulfur) prepolymer (bottom spectrum), and after an iterative copolymerization of this prepolymer with n-butyl acrylate (top spectrum), which clearly shows evidence for the formation of a poly(styrene-random-sulfur-n-butyl acrylate) terpolymer.

FIG. 3 shows non-limiting examples of unsaturated comonomers that can react with the sulfur prepolymer, and a non-limiting example of a stack plot of size exclusion chromatograms showing a poly(styrene-random-sulfur) prepolymer that, after iterative copolymerizations with di-allylic and tri-allylic comonomers, affords new polymers of significantly higher molecular weight.

FIG. 4 shows a non-limiting example of an unsaturated comonomer that can react with the sulfur prepolymer. FIG. 4 also shows a non-limiting example of comparative differential scanning calorimetry thermograms of poly(styrene-random-sulfur) prepolymer (top thermogram) and after iterative copolymerization with tri-allylic comonomer (bottom thermogram), which demonstrates enhancement of glass transition.

FIG. 5 shows another reaction scheme of dynamic covalent polymerization and examples of cross linkers.

FIG. 6 shows a comparison of non-iterative and iterative polymerization.

FIG. 7A shows inverse vulcanization and dynamic polymerization of sulfur-based polyols.

FIG. 7B shows dynamic polymerization of the sulfur-based polyol to produce the sulfur polyurethanes (SPU).

FIG. 7C shows dynamic polymerization of the sulfur polyurethanes to produce segmented SPUs.

FIG. 8A shows dynamic polymerization of the sulfur-based DIB polyol to produce a sulfur terpolymer.

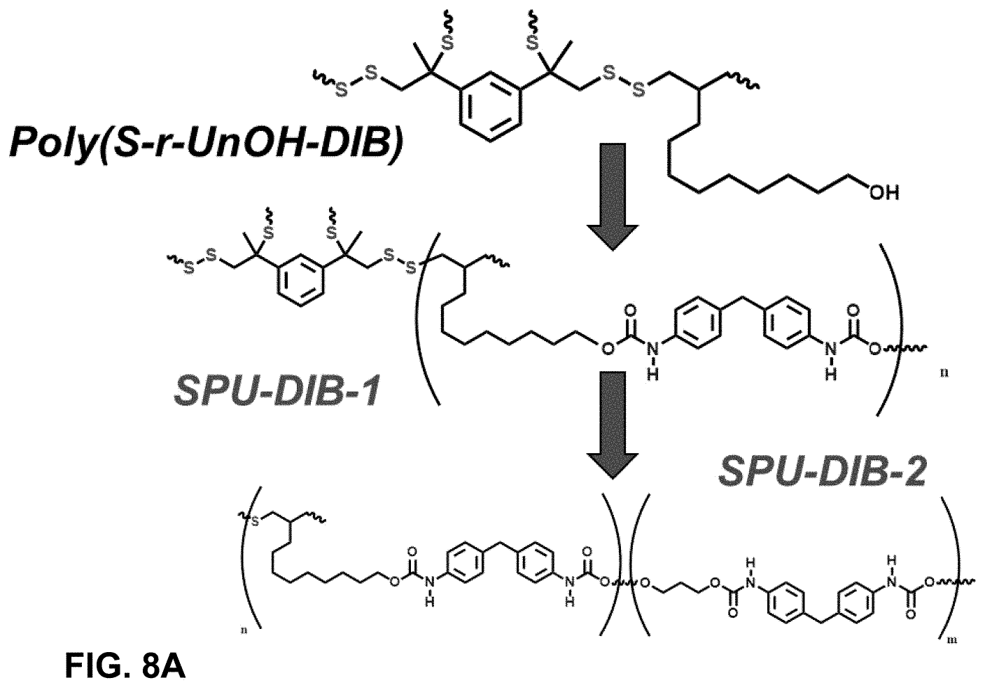

FIG. 8B shows the SEC of the sulfur-based DIB polyol, SPU-DIB-1 Prepolymer, and SPU-DIB-2 Segmented.

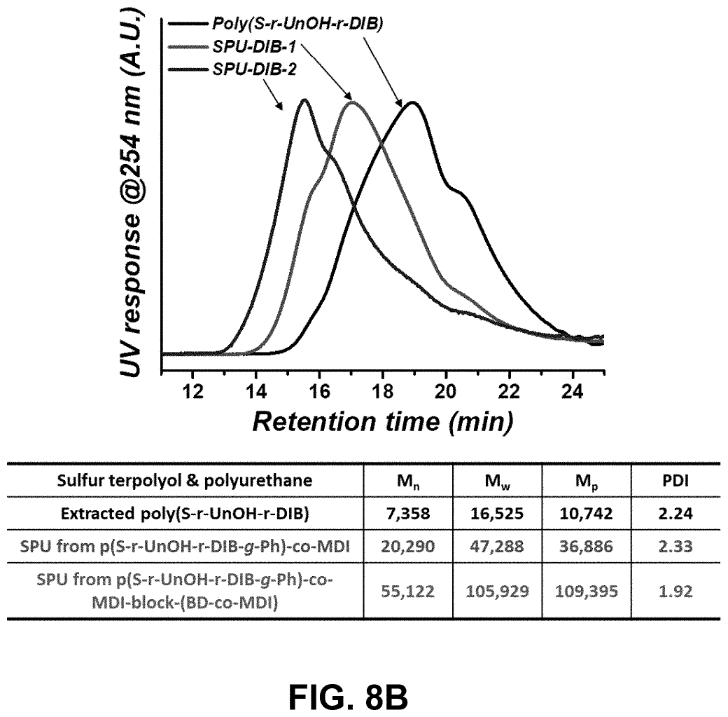

FIG. 9A shows another dynamic polymerization of the sulfur-based PDMS polyol to produce a sulfur terpolymer.

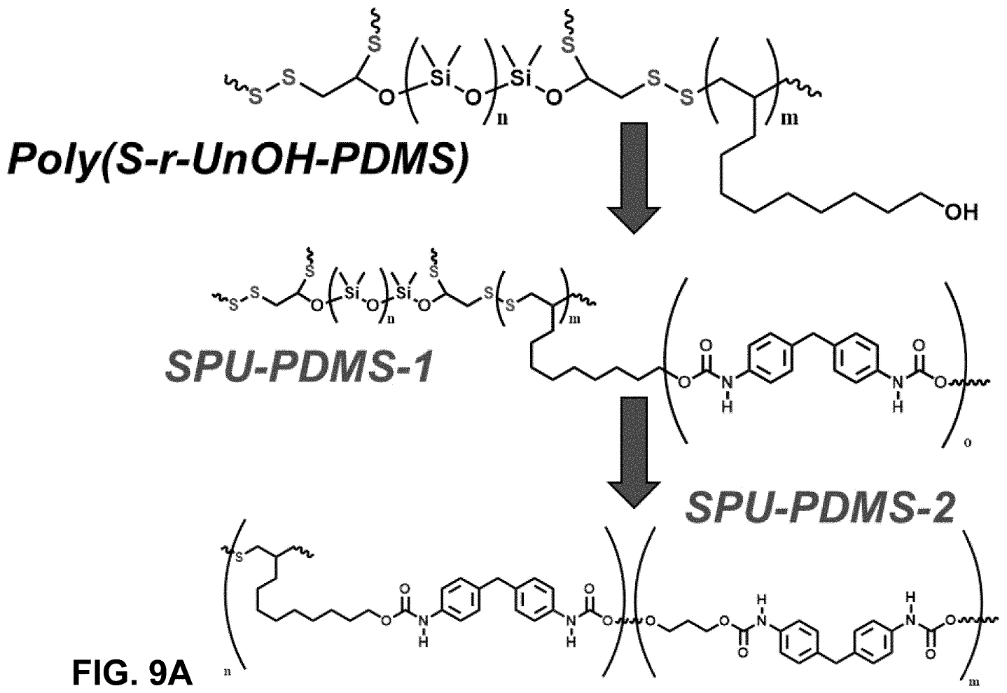

FIG. 9B shows the SEC of sulfur-based PDMS polyol, SPU-PDMS-1 Prepolymer, and SPU-DIB-2 Segmented.

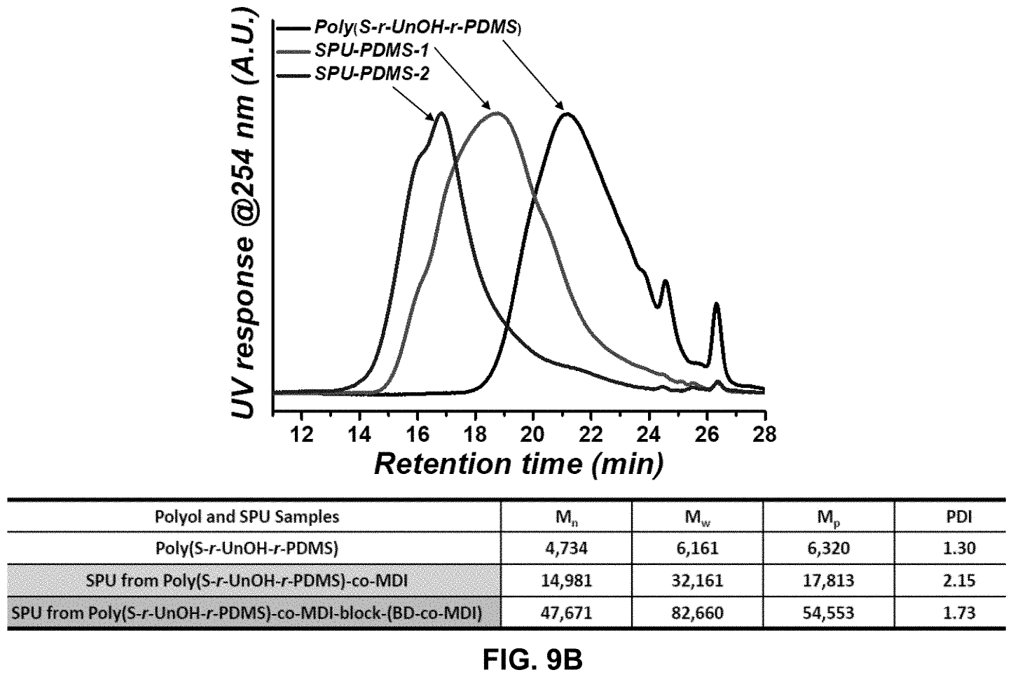

FIG. 10 shows an example of a substrate made from sulfur based polyurethanes.

### DESCRIPTION OF PREFERRED EMBODIMENTS

As used herein, sulfur can be provided as elemental sulfur, for example, in powdered form. Under ambient conditions, elemental sulfur primarily exists in an eight-membered ring form (S₈) which melts at temperatures in the range of 120° C.-130° C. and undergoes an equilibrium ring-opening polymerization (ROP) of the S₈ monomer into a linear polysulfane with diradical chain ends, referred to herein as a sulfur monomer. As the person of skill in the art will appreciate, while S₈ is generally the most stable, most accessible and cheapest feedstock, many other allotropes of sulfur can be used (such as other cyclic allotropes, derivable by melt-thermal processing of S₈). Any sulfur species that yield diradical or anionic polymerizing species when heated as described herein can be used in practicing the present invention.

As used herein, the term “sulfur polymer” generally refers to any polymer that contains sulfur monomers. Sulfur polymer may be used interchangeably with sulfur polymer composition, or sulfur terpolymer, unless specified otherwise.

As used herein, a “styrenic comonomer” is a monomer that has a vinyl functional group. The styrenic comonomer may comprise a styrene and at least one reactive functional group. As known to one of ordinary skill in the art, a styrene is a derivative of benzene ring that has a vinylic moiety. The sulfur diradicals can link to the vinylic moieties of the styrenic commoners to form the sulfur-styrenic polymer. In certain embodiments, the reactive functional group may be a halogen, an alkyl halide, an alkyl, an alkoxy, an amine, or a nitro functional group. Non-limiting examples of styrenic comonomers include bromostyrene, chlorostyrene, (trifluoromethyl)styrene, fluorostyrene, vinylaniline, acetoxystyrene, methoxystyrene, ethoxystyrene, methylstyrene, nitrostyrene, vinylbenzoic acid, vinylanisole, and vinylbenzyl chloride.

As used herein, the term “amine monomer” is a monomer that has an amine functional group. In one embodiment, aromatic amines and multi-functional amines may be used. Amine monomers include, but are not limited to, aromatic amines, vinylaniline, m-phenylenediamine, and p-phenylenediamine. The various types of phenylenediamines are inexpensive reagents due to their wide-spread use in the preparation of many conventional polymers, e.g., polyureas, polyamides.

As used herein, the term “thiol monomer” is a monomer that has a thiol functional group. Thiol monomers include, but are not limited to, 4,4′-thiobisbenzenethiol and the like. The term “sulfide monomers” are monomers that have sulfide functional groups.

As used herein, the term “nitrone monomer” is a monomer that has a nitrone group. In one embodiment, nitrones, dinitrones, and multi-nitrones may be used. Examples include, but are not limited to, N-benzylidene-2-methylpropan-2-amine oxide.

As used herein, an “aldehyde monomer” is a monomer that has an aldehyde functional group. In one embodiment, aldehydes, dialdehydes, and multi-aldehydes may be used.

As used herein, the term “ketone monomer” is a monomer that has a ketone functional group. In one embodiment, ketones, di-ketones, and multi-ketones may be used.

As used herein, the term “epoxide monomer” is a monomer that has epoxide functional groups. Non-limiting examples of such monomers include, generally, mono- or polyoxiranylbenzenes, mono- or polyglycidylbenzenes, mono- or polyglycidyloxybenzenes, mono- or polyoxiranyl(hetero)aromatic compounds, mono-or polyglycidyl(hetero)aromatic compounds, mono- or polyglycidyloxy(hetero)aromatic compounds, diglycidyl bisphenol A ethers, mono- or polyglycidyl(cyclo)alkyl ethers, mono- or polyepoxy(cyclo)alkane compounds and oxirane-terminated oligomers. In one preferred embodiment, the epoxide monomers may be benzyl glycidyl ether and tris(4-hydroxyphenyl)methane triglycidyl ether. In certain embodiments, the epoxide monomers may include a (hetero)aromatic moiety such as, for example, a phenyl, a pyridine, a triazine, a pyrene, a naphthalene, or a polycyclic (hetero)aromatic ring system, bearing one or more epoxide groups. For example, in certain embodiments, the one or more epoxide monomers are selected from epoxy(hetero)aromatic compounds, such as styrene oxide and stilbene oxide and (hetero)aromatic glycidyl compounds, such as glycidyl phenyl ethers (e.g., resorcinol diglycidyl ether, glycidyl 2-methylphenyl ether), glycidylbenzenes (e.g., (2,3-epoxypropyl)benzene) and glycidyl heteroaromatic compounds (e.g., N-(2,3-epoxypropyl)phthalimide). In certain desirable embodiments, an epoxide monomer will have a boiling point greater than 180° C., greater than 200° C., or even greater than 230° C. at the pressure at which polymerization is performed (e.g., at standard pressure, or at other pressures).

As used herein, the term “thiirane monomer” is a monomer that has a thirane functional group. Non-limiting examples of thiirane monomers include, generally, mono- or polythiiranylbenzenes, mono- or polythiiranylmethylbenzenes, mono- or polythiiranyl(hetero)aromatic compounds, mono- or polythiiranylmethyl(hetero)-aromatic compounds, dithiiranylmethyl bisphenol A ethers, mono- or polydithiiranyl (cyclo)alkyl ethers, mono- or polyepisulfide(cyclo)alkane compounds, and thiirane-terminated oligomers. In some embodiments, thiirane monomers may include a (hetero)aromatic moiety such as, for example, a phenyl, a pyridine, a triazine, a pyrene, a naphthalene, or a poly cyclic (hetero)aromatic ring system, bearing one or more thiirane groups. In certain desirable embodiments, a thiirane monomer can have a boiling point greater than 180° C., greater than 200° C., or even greater than 230° C. at the pressure at which polymerization is performed (e.g., at standard pressure).

As used herein, an ethylenically unsaturated monomer is a monomer that contains an ethylenically unsaturated functional group (i.e. double bond). The term “ethylenically unsaturated” may be used interchangeably with the term “unsaturated”. One of ordinary skill in the art will understand that “unsaturated” refers to the C═C functional group. The term “ethylenically unsaturated monomer” does not include compounds in which the ethylenic unsaturation is part of a long chain alkyl moiety (e.g. unsaturated fatty acids such as oleates, and unsaturated plant oils).

Non-limiting examples of ethylenically unsaturated monomers include vinyl monomers, acryl monomers, (meth)acryl monomers, unsaturated hydrocarbon monomers, and ethylenically-terminated oligomers. Examples of such monomers include, generally, mono- or polyvinylbenzenes, mono- or polyisopropenylbenzenes, mono- or polyvinyl(hetero)aromatic compounds, mono- or polyisopropenyl(hetero)-aromatic compounds, acrylates, methacrylates, alkylene di(meth)acrylates, bisphenol A di(meth)acrylates, benzyl (meth)acrylates, phenyl(meth)acrylates, heteroaryl (meth)acrylates, terpenes (e.g., squalene) and carotene. In some embodiments, non-limiting examples of ethylenically unsaturated monomers that are non-homopolymerizing include allylic monomers, isopropenyls, maleimides, norbornenes, vinyl ethers, and methacrylonitrile. In other embodiments, the ethylenically unsaturated monomers may include a (hetero)aromatic moiety such as, for example, phenyl, pyridine, triazine, pyrene, naphthalene, or a polycyclic (hetero)aromatic ring system, bearing one or more vinylic, acrylic or methacrylic substituents. Examples of such monomers include benzyl (meth)acrylates, phenyl (meth)acrylates, divinylbenzenes (e.g., 1,3-divinylbenzene, 1,4-divinylbenzene), isopropenyl-benzene, styrenics (e.g., styrene, 4-methylstyrene, 4-chlorostyrene, 2,6-dichlorostyrene, 4-vinylbenzyl chloride), diisopropenylbenzenes (e.g., 1,3-diisopropenylbenzene), vinylpyridines (e.g., 2-vinylpyridine, 4-vinylpyridine), 2,4,6-tris((4-vinylbenzyl)thio)-1,3,5-triazine and divinylpyridines (e.g., 2,5-divinylpyridine). In certain embodiments, the ethylenically unsaturated monomers (e.g., including an aromatic moiety) bear an amino (i.e., primary or secondary) group, a phosphine group or a thiol group. One example of such a monomer is vinyldiphenylphosphine. In certain desirable embodiments, an ethylenically unsaturated monomer will have a boiling point greater than 180° C., greater than 200° C., or even greater than 230° C. at the pressure at which polymerization is performed (e.g., at standard pressure).

As used herein, an alkynylly unsaturated monomer is a monomer that has an alkynylly unsaturated functional group (i.e. triple bond). The term “alkynylly unsaturated monomer” does not include compounds in which the alkynyl unsaturation is part of a long chain alkyl moiety (e.g., unsaturated fatty acids, or carboxylic salts, or esters such as oleates, and unsaturated plant oils). In one embodiment, aromatic alkynes, both internal and terminal alkynes, multi-functional alkynes may be used. Examples of alkynylly unsaturated monomers include, but are not limited to, ethynylbenzene, 1-phenylpropyne, 1,2-diphenylethyne, 1,4-diethynylbenzene, 1,4-bis(phenylethynyl)-benzene, and 1,4-diphenylbuta-1,3-diyne.

As used herein, the term “self-healing” refers to the ability to repair damage with minimum intervention. The mechanisms and techniques to enable self-healing may include covalent bonding, supramolecular chemistry, H-bonding, ionic interactions, π-π stacking, chemo-mechanical repairs focusing on encapsulation, remote self-healing, or shape memory assisted polymers. In one embodiment, self-healing utilizes thermal reformation, which involves the use of heat to reform the bonds or cross-links of a polymeric material. As used herein, the term “dynamic” is defined as the reversibly breaking of bonds.

As used herein, the term “functional” in correlation with a polymer refers to functional polymers that have specified physical, chemical, biological, pharmacological, or other properties or uses that are determined by the presence of specific chemical functional groups.

As used herein, the terms “those defined above” and “those defined herein” when referring to a variable incorporates by reference the broad definition of the variable as well as any narrow and/or preferred definitions, if any.

Most organic monomers are immiscible with liquid sulfur. The present invention resolves this problem by first making a liquid sulfur prepolymer from inverse vulcanization with S8 and a first monomer, and then adding a second monomer that would otherwise be immiscible with liquid sulfur.

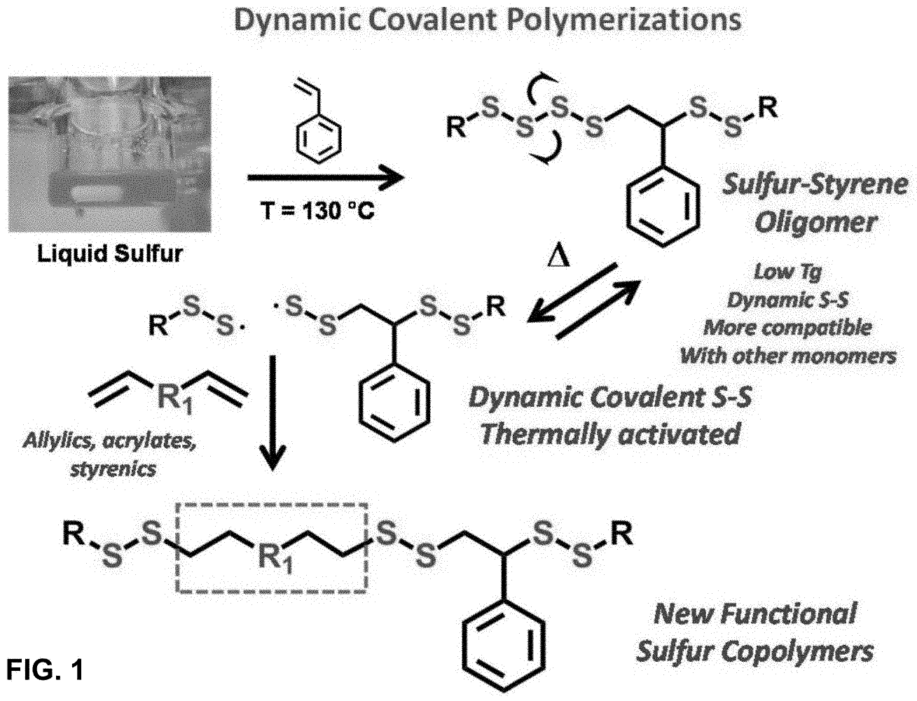

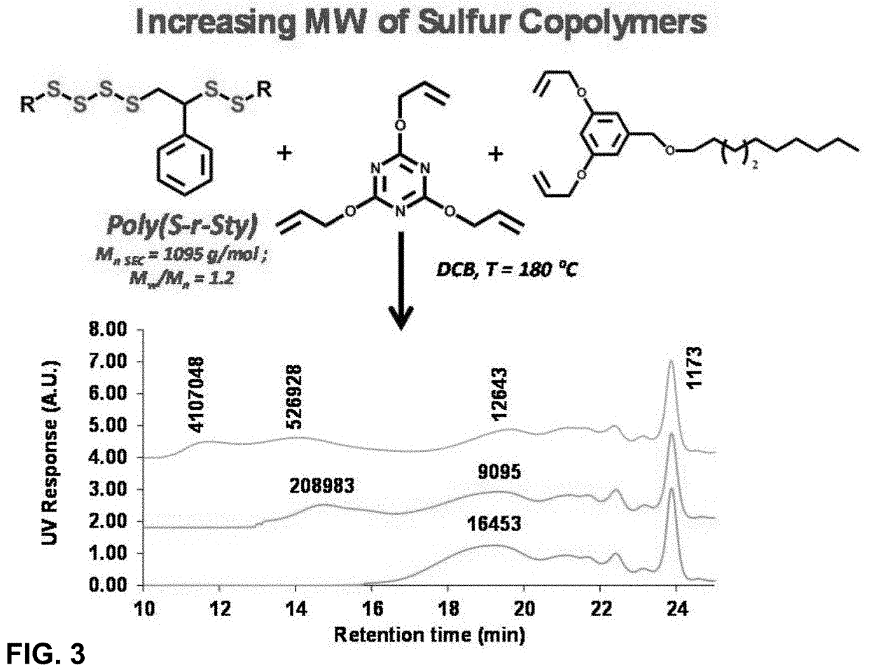

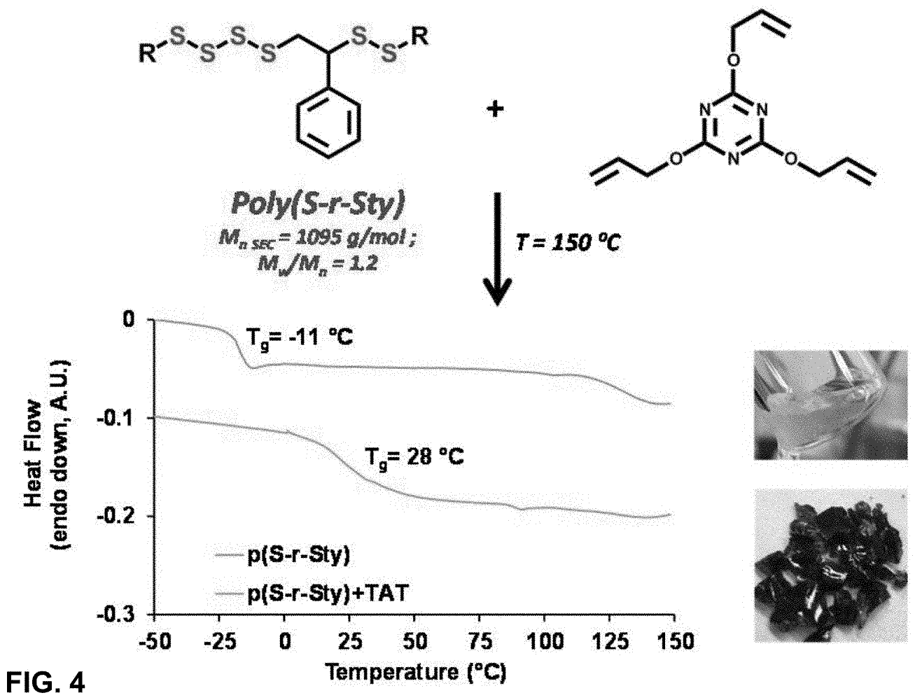

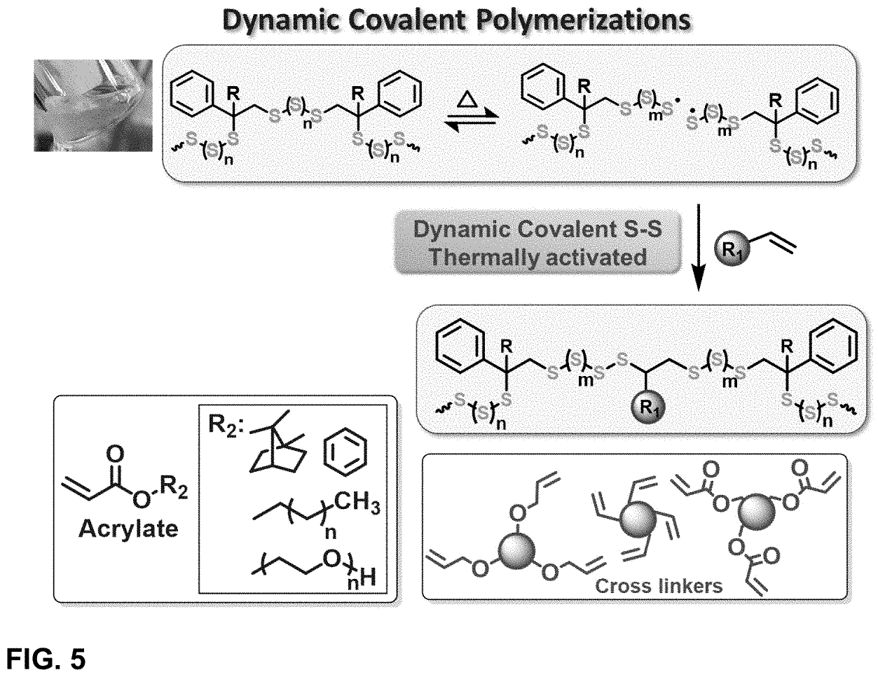

Referring now to FIG. 1-7B, it is an objective of the present invention to provide an iterative method that improves processability of elemental sulfur in producing a desired sulfur polymer by initially copolymerizing the elemental sulfur with first comonomers to prepare a sulfur prepolymer that is tailored to be miscibly compatible with second comonomers. According to one embodiment, the iterative method of synthesizing the sulfur polymer may comprise providing elemental sulfur and heating the elemental sulfur to form a molten sulfur comprising dynamic sulfur-sulfur (S—S) bonds. Without wishing to limit the invention to a particular theory or mechanism, the heating enables thermal activation and scission of the dynamic S—S bonds from said molten sulfur to form sulfur radicals. It is further theorized that the dynamic S—S bonds are relatively weak sulfur bonds that break upon thermal activation.

In some embodiments, the method may further comprise selecting one or more first comonomers, each first comonomer comprising at least one reactive functional group and adding the first comonomers to the molten sulfur. Without wishing to limit the invention to a particular theory or mechanism, the first comonomers and molten sulfur copolymerize by a reaction of the sulfur radicals with the reactive functional groups of the first comonomers to form a sulfur prepolymer. In other embodiments, the method may further comprise selecting one or more second comonomers such that the sulfur prepolymer is miscibly compatible with the second comonomers, where each of the second comonomers comprise at least one reactive functional group, heating the sulfur prepolymer to form a molten sulfur prepolymer comprising the dynamic S—S bonds, and adding the second comonomers to the molten sulfur prepolymer, where the molten sulfur prepolymer is miscible with the second comonomers. Without wishing to limit the invention to a particular theory or mechanism, the heating enables thermal activation and scission of the dynamic S—S bonds from said molten sulfur prepolymer to form sulfur-containing radical species. Further still, the second comonomers and the sulfur prepolymer copolymerize by a reacting of the sulfur-containing radical species with the reactive functional groups of the second comonomers to form the sulfur polymer.

In preferred embodiments, the first comonomers may be selected to form the sulfur prepolymer having a tunable composition that enables miscibility or solubility of the sulfur prepolymer with the second comonomers to produce complex sulfur polymers. In some preferred embodiments, the second comonomers may be selected such that the second comonomers are soluble in the molten sulfur prepolymer. In other preferred embodiments, the selection of the second comonomers may further be determined by a number of reactive functional groups of each second comonomer. Without wishing to limit the invention to a particular theory or mechanism, the second comonomers that have an increasing number of reactive functional groups can further enable branching or crosslinking in the sulfur polymer.

In some embodiments, the elemental sulfur may be about 10-90 wt % of the sulfur polymer. For example, the elemental sulfur may be at least 50 wt % of the sulfur polymer. In other embodiments, the elemental sulfur may be about 10 to 30 wt %, 30 to 40 wt %, 40 to 60 wt %, 60 to 80 wt %, or 80 to 95 wt % of the polymer.

In some embodiments, the first comonomers may be about 5-50 wt % of the sulfur polymer. For example, the first comonomers may be at most about 50 wt % of the sulfur polymer. In other embodiments, the first comonomers may be about 5 to 10 wt %, 10 to 20 wt %, 20 to 30 wt %, 30 to 40 wt %, or 40 to 50 wt % of the polymer. In some embodiments, the first comonomers may be selected from a group consisting of amine comonomers, thiol comonomers, sulfide comonomers, alkynylly unsaturated comonomers, epoxide comonomers, nitrone comonomers, aldehyde comonomers, ketone comonomers, thiirane comonomers, ethylenically unsaturated comonomers, styrenic comonomers, vinylic comonomers, methacrylate comonomers, acrylonitrile comonomers, allylic monomers, acrylate monomers, vinylpyridine monomers, isobutylene monomers, maleimide monomers, norbornene monomers, monomers having at least one vinyl ether moiety, and monomers having at least one isopropenyl moiety.

In some embodiments, the second comonomers may be about 5-50 wt % of the sulfur polymer. For example, the second comonomers may be at most about 50 wt % of the polymer. In still other embodiments, the second comonomers may be about 5 to 10 wt %, 10 to 20 wt %, 20 to 30 wt %, 30 to 40 wt %, or 40 to 50 wt % of the polymer. In some embodiments, the second comonomers may be selected from a group consisting of amine comonomers, thiol comonomers, sulfide comonomers, alkynylly unsaturated comonomers, epoxide comonomers, nitrone comonomers, aldehyde comonomers, ketone comonomers, thiirane comonomers, ethylenically unsaturated comonomers, styrenic comonomers, vinylic comonomers, methacrylate comonomers, acrylonitrile comonomers, allylic monomers, acrylate monomers, vinylpyridine monomers, isobutylene monomers, maleimide monomers, norbornene monomers, monomers having at least one vinyl ether moiety, and monomers having at least one isopropenyl moiety.

It is another objective of the present invention to provide an iterative method that improves processability of elemental sulfur in producing a desired sulfur polymer by initially copolymerizing the elemental sulfur with the unsaturated comonomers to prepare the sulfur prepolymer that is tailored to be miscibly compatible with the second comonomers. According to one embodiment, the iterative method of synthesizing the sulfur polymer may comprise providing elemental sulfur and heating the elemental sulfur to form a molten sulfur comprising dynamic sulfur-sulfur (S—S) bonds. Without wishing to limit the invention to a particular theory or mechanism, the heating enables thermal activation and scission of the dynamic S—S bonds from said molten sulfur to form sulfur radicals. It is further theorized that the dynamic S—S bonds are relatively weak sulfur bonds that break upon thermal activation.

In one embodiment, the method may further comprise selecting one or more unsaturated comonomers, each unsaturated comonomer comprising at least one alkenyl functional group and adding the unsaturated comonomers to the molten sulfur. Without wishing to limit the invention to a particular theory or mechanism, the unsaturated comonomers and molten sulfur copolymerize by a reaction of the sulfur radicals with the alkenyl functional groups to form a sulfur prepolymer. For instance, the copolymerization of the unsaturated comonomers and molten sulfur occurs via a thiol-ene reaction or other related processes.

In another embodiment, the method may further comprise selecting one or more second comonomers such that the sulfur prepolymer is miscibly compatible with the second comonomers, where each of the second comonomers comprise at least one reactive functional group, heating the sulfur prepolymer to form a molten sulfur prepolymer comprising the dynamic S—S bonds, and adding the second comonomers to the molten sulfur prepolymer, where the molten sulfur prepolymer is miscible with the second comonomers. Without wishing to limit the invention to a particular theory or mechanism, the heating enables thermal activation and scission of the dynamic S—S bonds from said molten sulfur prepolymer to form sulfur-containing radical species. It is further theorized that the second comonomers and the sulfur prepolymer copolymerize by a reacting of the sulfur-containing radical species with the reactive functional groups of the second comonomers to form the sulfur polymer. For example, the copolymerization of the second comonomers and the sulfur prepolymer occurs via a thiol-ene reaction or other related processes.

While thiol-ene reactions, or alkene hydrothiolation, are known to one of ordinary skill in the art, for illustrative purposes, the mechanisms thereof involve the reaction of the sulfur radicals with the C—C double bond of the comonomer to form an S—C bond. The person of skill in the art will select conditions that provide the desired level of polymerization. In certain embodiments, the polymerization reaction is performed under ambient pressure. However, in other embodiments, the polymerization reaction can be performed at elevated pressure (e.g., in a bomb or an autoclave). Elevated pressures can be used to polymerize more volatile comonomers, so that they do not vaporize under the elevated temperature reaction conditions.

In a preferred embodiment, the unsaturated comonomers may be selected to form the sulfur prepolymer having a tunable composition that enables miscibility or solubility of the sulfur prepolymer with the second comonomers to produce complex sulfur polymers. In another preferred embodiment, the second comonomers may be selected such that the second comonomers are soluble in the molten sulfur prepolymer. In yet another preferred embodiment, the selection of the second comonomers is further determined by a number of reactive functional groups of each second comonomer. Without wishing to limit the invention to a particular theory or mechanism, the second comonomers having an increasing number of reactive functional groups further enables branching or crosslinking in the sulfur polymer.

In one embodiment, the elemental sulfur may be about 10-90 wt % of the sulfur polymer. For example, the elemental sulfur may be at least 50 wt % of the sulfur polymer. In another embodiment, the elemental sulfur may be about 10 to 30 wt %, 30 to 40 wt %, 40 to 60 wt %, 60 to 80 wt %, or 80 to 95 wt % of the polymer.

In another embodiment, the unsaturated comonomers may be about 5-50 wt % of the sulfur polymer. For example, the unsaturated comonomers may be at most about 50 wt % of the sulfur polymer. In yet another embodiment, the unsaturated comonomers may be about 5 to 10 wt %, 10 to 20 wt %, 20 to 30 wt %, 30 to 40 wt %, or 40 to 50 wt % of the polymer. In preferred embodiments, the at least one alkenyl functional group of the unsaturated comonomers may be a vinylic functional group. Non-limiting examples of the unsaturated comonomers include styrenic comonomers, allylic comonomers, methacrylate comonomers, acrylate comonomers, and norbornene comonomers.

In another embodiment, the second comonomers may be about 5-50 wt % of the sulfur polymer. For example, the second comonomers may be at most about 50 wt % of the polymer. In still other embodiments, the second comonomers may be about 5 to 10 wt %, 10 to 20 wt %, 20 to 30 wt %, 30 to 40 wt %, or 40 to 50 wt % of the polymer. In some embodiments, the second comonomers may be selected from a group consisting of amine comonomers, thiol comonomers, sulfide comonomers, alkynylly unsaturated comonomers, epoxide comonomers, nitrone comonomers, aldehyde comonomers, ketone comonomers, thiirane comonomers, ethylenically unsaturated comonomers, styrenic comonomers, vinylic comonomers, methacrylate comonomers, acrylonitrile comonomers, allylic monomers, acrylate monomers, vinylpyridine monomers, isobutylene monomers, maleimide monomers, norbornene monomers, monomers having at least one vinyl ether moiety, and monomers having at least one isopropenyl moiety.

Consistent with any of the methods described herein, other examples of the first or second comonomers include, but are not limited to, DIB and analogues, alkynes, alkenes, unsaturated, cyclic saturated, aliphatic compounds with unsaturation, methacrylates, acrylates, dienes, polydienes, acrylonitrile, vinyl ethers, vinyl esters and functional vinylic comonomers. In still other embodiments, the ethylenically unsaturated comonomers are comonomers that are capable of thiol-ene, or thiol-yne reactions.

In accordance with any of the methods described herein, the elemental sulfur may be melted at a temperature of about 120-140° C. For example, the melt temperature may be about 130° C. In some embodiment, the sulfur prepolymer may be heated to a temperature of about 140° C.-200° C. For example, the sulfur prepolymer may be heated to a temperature of about 150° C.-180° C., or about 160° C.-170° C.

In other embodiments, the methods described herein may further comprise adding a solvent to the sulfur prepolymer prior to heating the sulfur prepolymer. For example, the solvent may comprise an organic solvent, such as aromatic solvent, toluene, THF, DMSO, and DMF, a diluent, an aqueous medium, or a combination thereof. The solvent can dilute the sulfur prepolymer prior to heating. In still other embodiments, the methods described herein may be performed in a solvent. For example, the solvent may comprise an organic solvent, such as aromatic solvent, toluene, THF, DMSO, and DMF, a diluent, an aqueous medium, or a combination thereof.

Non-limiting examples of polymerization reactions that may occur include free radical polymerization, controlled radical polymerization, ring-opening polymerization, ring-opening metathesis polymerization, step-growth polymerization, and chain-growth polymerization.

In still other embodiments, the sulfur polymer may be further reacted with other comonomers. For instance, a sulfur-containing radical or an available reactive functional group of the functional sulfur polymer may react with the reactive functional group of the other comonomers. The technique of reacting can be oxidative coupling, free radical polymerization, or copolymerization.

While methods of synthesizing sulfur polymers have been described, it is another objective of the invention to provide high sulfur content polymers. Thus, in some embodiments, the present invention features a sulfur polymer composition comprising a copolymerized product of a sulfur prepolymer and about 5-50 wt % of one or more second comonomers, each second comonomer having at least one reactive functional group. Without wishing to be bound by theory, the sulfur prepolymer is miscibly compatible with the second comonomers.

In one embodiment, the sulfur prepolymer may comprise a copolymerized product of at least about 50 wt % of elemental sulfur, and about 5-50 wt % of one or more first comonomers, each first comonomer having at least one reactive functional group. Without wishing to limit the invention to a particular theory or mechanism, the elemental sulfur is copolymerized with the first comonomers by a reaction of sulfur radicals derived from the elemental sulfur with the reactive functional groups of the first comonomers to form the sulfur prepolymer. In some embodiments, the first comonomers may be selected from a group consisting of amine comonomers, thiol comonomers, sulfide comonomers, alkynylly unsaturated comonomers, epoxide comonomers, nitrone comonomers, aldehyde comonomers, ketone comonomers, thiirane comonomers, ethylenically unsaturated comonomers, styrenic comonomers, vinylic comonomers, methacrylate comonomers, acrylonitrile comonomers, allylic monomers, acrylate monomers, vinylpyridine monomers, isobutylene monomers, maleimide monomers, norbornene monomers, monomers having at least one vinyl ether moiety, and monomers having at least one isopropenyl moiety.

In another embodiment, the sulfur prepolymer may comprise a copolymerized product of at least about 50 wt % of elemental sulfur, and about 5-50 wt % of one or more unsaturated comonomers, each unsaturated comonomer having at least one alkenyl functional group. Without wishing to limit the invention to a particular theory or mechanism, the elemental sulfur is copolymerized with the unsaturated comonomers by a thiol-ene reaction of sulfur radicals derived from the elemental sulfur and the alkenyl functional groups of the unsaturated comonomers to form the sulfur prepolymer. In some embodiments, the alkenyl functional groups of the unsaturated comonomers are vinylic functional groups. In other embodiments, the unsaturated comonomers may be styrenic comonomers, allylic comonomers, methacrylate comonomers, acrylate comonomers, and norbornene comonomers.

While in some embodiments, the second comonomers may have at least one reactive functional group; in other embodiments, the second comonomers may have a plurality of reactive functional groups. Without wishing to limit the invention to a particular theory or mechanism, the plurality of reactive functional groups may enable branching or crosslinking in the sulfur polymer to form a complex polymer material.

In accordance with previous embodiments, the second comonomers may be amine comonomers, thiol comonomers, sulfide comonomers, alkynylly unsaturated comonomers, epoxide comonomers, nitrone comonomers, aldehyde comonomers, ketone comonomers, thiirane comonomers, ethylenically unsaturated comonomers, styrenic comonomers, vinylic comonomers, methacrylate comonomers, acrylonitrile comonomers, allylic monomers, acrylate monomers, vinylpyridine monomers, isobutylene monomers, maleimide monomers, norbornene monomers, monomers having at least one vinyl ether moiety, and monomers having at least one isopropenyl moiety.

Consistent with previous embodiments, other examples of the first or second comonomers include, but are not limited to, DIB and analogues, alkynes, alkenes, unsaturated, cyclic saturated, aliphatic compounds with unsaturation, methacrylates, acrylates, dienes, polydienes, acrylonitrile, vinyl ethers, vinyl esters and functional vinylic comonomers. In still other embodiments, the ethylenically unsaturated comonomers are comonomers that are capable of thiol-ene, or thiol-yne reactions

Without wishing to be bound by theory, the sulfur prepolymer is copolymerized with the second comonomers by a reaction of sulfur-containing radicals derived from the sulfur prepolymer with the reactive functional groups of the second comonomers to form the sulfur polymer. Further still, the composition of the sulfur prepolymer is tunable such that the sulfur prepolymer is miscible or soluble with the second comonomers.

In one embodiment, the sulfur radicals may be derived from the elemental sulfur by thermal activation and scission of dynamic S—S bonds of said elemental sulfur. In another embodiment, the sulfur-containing radicals may be derived from the sulfur prepolymer by thermal activation and scission of dynamic S—S bonds of said sulfur prepolymer.

In preferred embodiments, the sulfur polymers and more complex polymer materials, such as in the form of cross-linked polymers or non-crosslinked, intractable polymers, can be reprocessed by stimuli activation of dynamic S—S bonds in the polymer. The introduction of S—S bonds into an intractable polymer material, or cross-linked polymer network, can allow for re-processing of the polymer material due to dynamic breaking of S—S bonds. In one embodiment, the sulfur polymers described herein are dynamic covalent polymers. The dynamic covalent polymers may comprise a terpolymer, or a more complex polymer having S—S bonds and some other polymer segment that is intractable, or cross-linked. Stimuli, such as thermal, light, or another form of stimuli, can induce dynamic activation of S—S bonds to enable re-processing, or melt processing of otherwise non-reversible, processable polymeric materials.

For illustrative purposes, the following are non-limiting examples of preparing the sulfur polymers of the present invention. Equivalents or substitutes are within the scope of the invention.

### EXAMPLE 1

### Sulfur-styrene copolymerization to form a sulfur oligomer of poly(sulfur-random-styrene) (Poly(S-r-Sty))

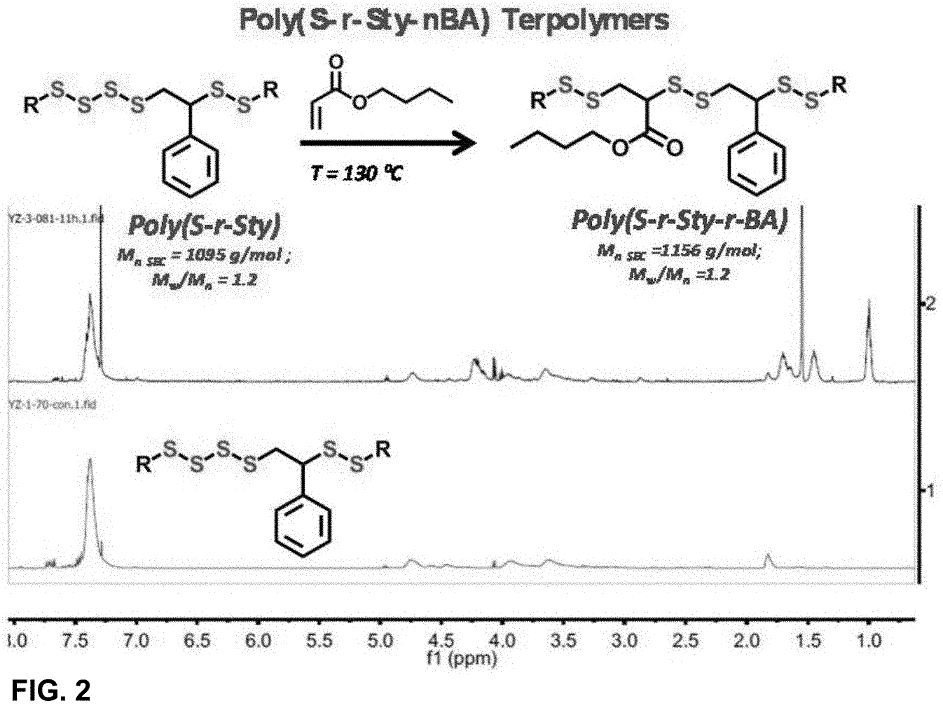

To a glass vial equipped a magnetic stir bar was added elemental sulfur and styrene and the reaction mixture was heated to 130° C. to afford the poly(S-r-Sty). The consumption of styrene was confirmed by 1H NMR spectra as shown in FIG. 2.

### EXAMPLE 2

### Poly(S-r-Sty) oligomer and n-butyl acrylate copolymerization to produce a sulfur polymer of poly(sulfur-random-styrene-random-n-butyl acrylate) (Poly(S-r-Sty-r-BA))

To a glass vial equipped a magnetic stir bar was added poly(S-r-Sty) and n-butyl acrylate and the reaction mixture was heated to 130° C. to afford the poly(S-r-Sty-r-BA).

### EXAMPLE 3

### Poly(S-r-Sty) oligomer and 2,4,6-Triallyloxy-1,3,5-triazine (TAT) copolymerization to produce a sulfur polymer

To a glass vial equipped a magnetic stir bar was added poly(S-r-Sty) and TAT and the reaction mixture was heated to 150° C. to afford the poly(S-r-Sty-r-BA)+TAT.

### EXAMPLE 4

### Comparison of iterative copolymerization

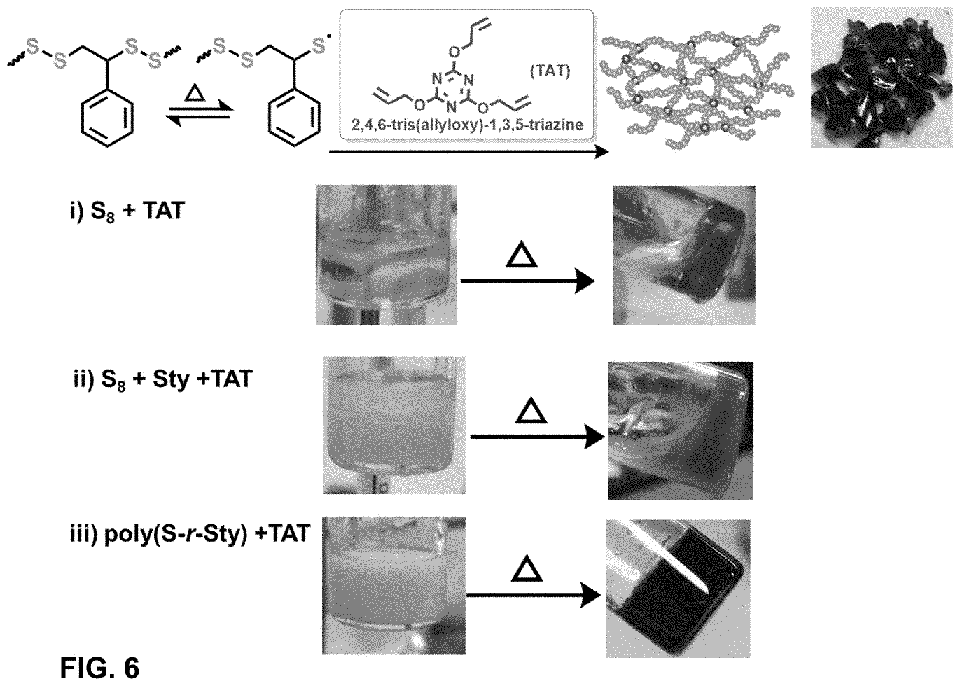

Referring to FIG. 6, scheme i) demonstrates that TAT monomer is immiscible in liquid sulfur and attempted polymerizations resulted in heterogeneous products. In scheme ii), mixing liquid sulfur, TAT, and styrene afforded immiscible fluids. Attempted polymerizations gave heterogeneous products. In scheme iii), using iterative copolymerization with the poly(S-r-Sty) resin and then adding TAT improves mixing. This process afforded homogeneous glassy copolymers with a single Tg indicated pure copolymer.

### EXAMPLE 5

### Sulfur based polyols

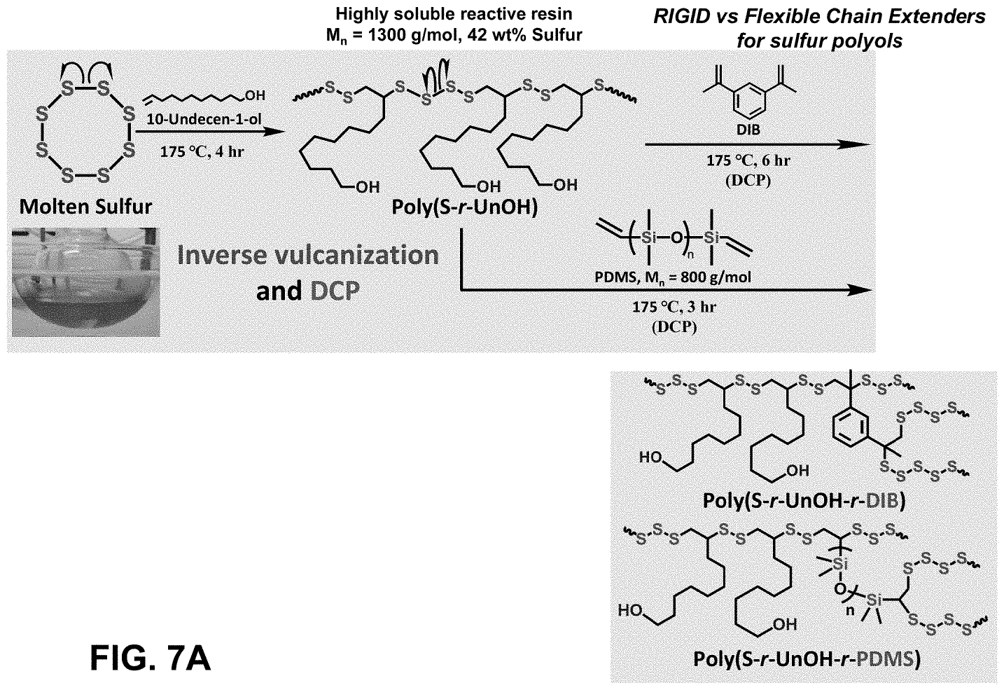

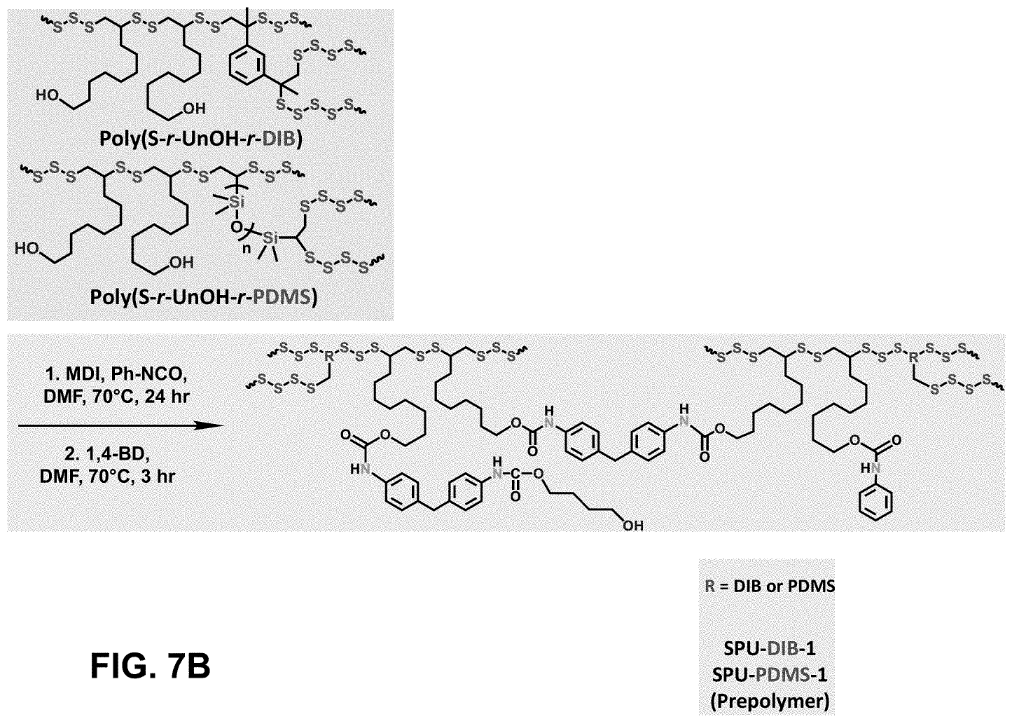

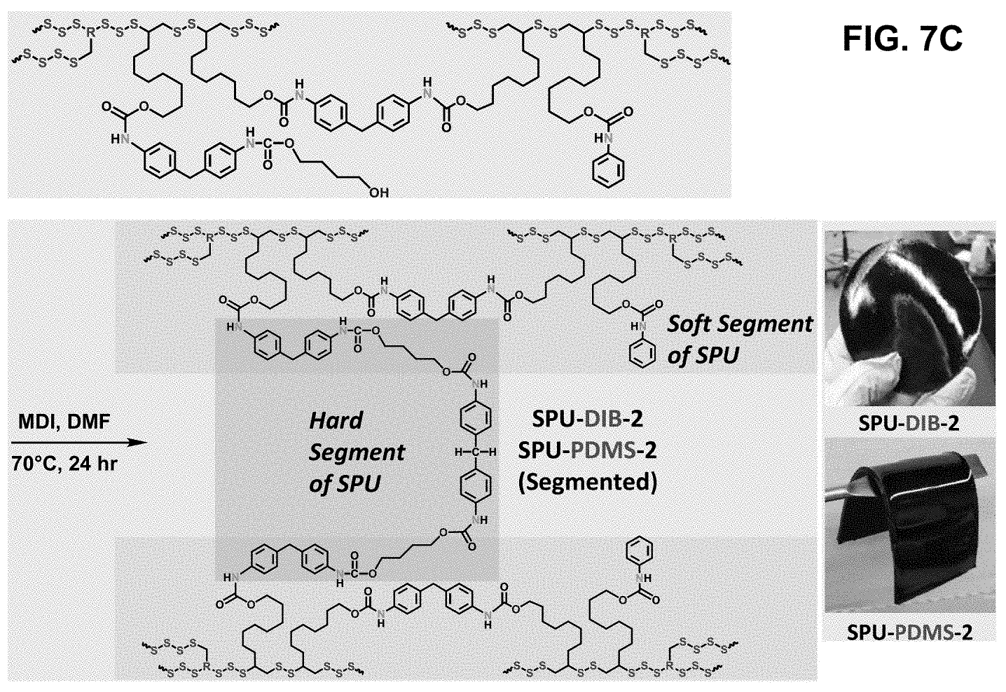

Referring to FIG. 7A, elemental sulfur and undecenol (UnOH) were mixed and heated to 175° C. to afford poly(S-r-UnOH). In one embodiment, poly(S-r-UnOH) and DIB were mixed and heated to afford poly(S-r-UnOH-r-DIB), which is a more rigid sulfur polyol. In another embodiment, poly(S-r-UnOH) and PDMS were mixed and heated to afford poly(S-r-UnOH-r-PDMS), which is a more flexible sulfur polyol.

In some embodiments, dynamic covalent polymerization (DCP) involves taking polymers made from inverse vulcanization from S8 and an unsaturated organic comonomer-1 that can be designed to carry an orthogonal functional group, such as alcohols. In other embodiments, the DCP step involves a second organic comomer-2 that has different hydrophobicity/hydrophilicity, and/or varying mechanical lossiness.

Referring to FIGS. 7B-7C, the present invention features a new class of segmented polyurethanes from sulfur. Without wishing to limit the present invention, these segmented polyurethanes have a tunable composition and improved mechanicals. The present invention demonstrates controlled molecular weight (MW) growth and polydispersity of soluble high MW polymer from elemental sulfur. These materials are also the first examples of a thermoplastic elastomer from sulfur and a flame retardant material from elemental sulfur.

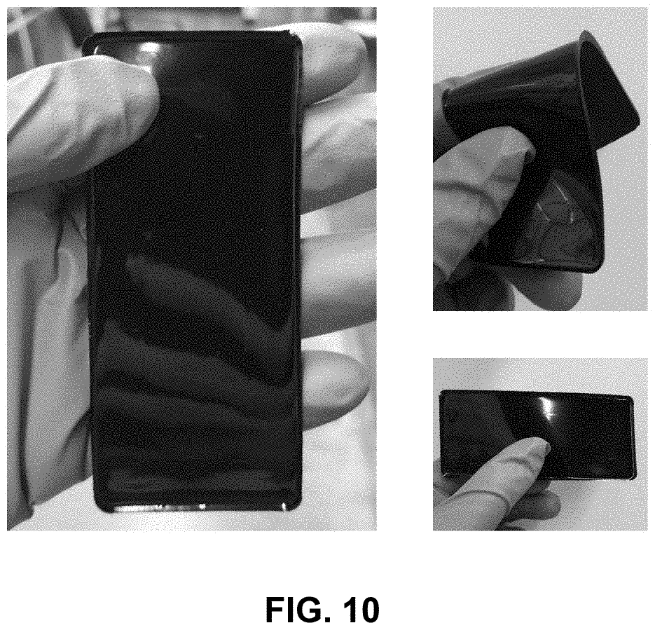

As shown in FIG. 10, the present invention provides segmented SPUs that can range from soft elastomers to strong ductile thermoplastics. No chromatography or recystallizations is needed for purification.

In some embodiments, the use of a new electroactive cathode material, which can be the sulfur polymer, for a Li—S battery, upon discharge, generates soluble additive species in situ that co-deposit onto the cathode with lower sulfide discharge products. These additive species may be introduced into the electroactive material during the synthesis of the material, or added to the electrolyte or battery separator as a soluble species. These additive species are able to co-deposit with sulfide-containing discharge products via active electrochemical reactions, or passive non-electrochemical processes. Co-deposition of these additive species with sulfide discharge products onto the Li—S cathode plasticizes the electrode against mechanical fracture during battery charge-discharge cycling. Plasticization enables retention of charge capacity and improve cycle lifetime beyond 100 cycles. The electroactive material in this case is best embodied by the sulfur polymers described herein. Upon discharge of this polymer, soluble organosulfur species are formed which function to improve Li—S batteries as described above.

An exemplary embodiment features an electrochemical cell comprising an anode comprising metallic lithium, a cathode comprising any of the aforementioned sulfur polymers that generate soluble additive species in situ upon discharge and the soluble additive species co-deposit with lower sulfide discharge products onto the cathode, and a non-aqueous electrolyte interposed between the cathode and the anode. In some embodiments, the lower sulfide discharge products are Li₂S₃, Li₂S₂, or Li₂S. Preferably, the electrochemical cell has an increased volumetric energy density. For example, the capacity of the electrochemical cell ranges from about 200 to about 1200 mAh/g.

Any embodiment of the electrochemical cells may be used in electric vehicle applications, portable consumer devices portable consumer devices (e.g., Personal electronics, cameras, electronic cigarettes, handheld game consoles, and flashlights), motorized wheelchairs, golf carts, electric bicycles, electric forklifts, tools, automobile starters, and uninterruptible power supplies.

In some embodiments, the electrolyte and/or a separator comprise the sulfur polymer. The sulfur polymer generates soluble organosulfur species upon discharge. The soluble additive species are co-deposited with the lower sulfide discharge products by an electrochemical reaction or a non-electrochemical reaction.

A person of skill in the art will select comonomers and relative ratios thereof in order to provide the desired properties to the polymer. In certain embodiments, the comonomers include one or more polyfunctional monomers, optionally or in combination with one or more monofunctional monomers. A polyfunctional monomer is one that includes more than one (e.g., 2, or 3) polymerizable functional groups such as amine, thiol, sulfide, alkynylly unsaturated, nitrone and/or nitroso, aldehyde, ketone, thiirane, ethylenically unsaturated, and/or epoxide moieties moieties. The multiple polymerizable groups of a polyfunctional monomer can be the same or different. A non-limiting example is a divinylbenzene monoxide. Polyfunctional monomers can be used to cross-link the sulfur or sulfur polymer chains to adjust the properties of the polymer, as would be understood by the person of skill in the art. Fréchet-type benzyl ether dendrimers bearing styrenic terminal groups are miscible with liquid sulfur and can be used as polyfunctional cross-linkers. In certain embodiments, the one or more polyfunctional monomers include one or more of a divinylbenzene, a diisopropenylbenzene, an alkylene di(meth)acrylate, a bisphenol A di(meth)acrylate, a terpene, a carotene, a divinyl (hetero)aromatic compound, and a diisopropenyl (hetero)aromatic compound.

Any desirable combination of the comonomer can be used in the same sulfur polymer. As non-limiting examples, in one embodiment of the invention, the first or second comonomers may be a combination of amine monomers and styrenic monomers.

Embodiments of the present invention featuring any of sulfur polymers described herein may further comprise an elemental carbon material, which may be dispersed in the sulfur polymer, in an amount of at most about 50 wt % of the sulfur polymer. In some embodiments, the methods of synthesizing any of the sulfur polymers described herein may further comprise the step of dispersing an elemental carbon material in the sulfur copolymers. For example, the carbon material is at most about 5 wt %, or at most about 10 wt %, or at most about 20 wt %, or at most about 30 wt %, or at most about 40 wt %, or at most about 50 wt % of the sulfur polymer.

Alternative embodiments of the sulfur polymer further comprise one or more monofunctional monomers, or one or more polyfunctional monomers (e.g., difunctional or trifunctional). The one or more polyfunctional monomers is selected from a group consisting of a polyvinyl monomer (e.g., divinyl, trivinyl), a polyisopropenyl monomer (e.g., diisoprenyl, triisoprenyl), a polyacryl monomer (e.g., diacryl, triacryl), a polymethacryl monomer (e.g., dimethacryl, trimethacryl), a polyunsaturated hydrocarbon monomer (e.g., diunsaturated, triunsaturated), a polyepoxide monomer (e.g., diepoxide, triepoxide), a polythiirane monomer (e.g., dithiirane, trithiirane), a polyalkynyl monomer, a polydiene monomer, a polybutadiene monomer, a polyisoprene monomer, a polynorbornene monomer, a polyamine monomer, a polythiol monomer, a polysulfide monomer, a polyalkynylly unsaturated monomers, a polynitrone monomers, a polyaldehyde monomers, a polyketone monomers, and a polyethylenically unsaturated monomers. In some embodiments, the one or more polyfunctional monomers is selected from a group consisting of a divinylbenzene, a diisopropenylbenzene, an alkylene di(meth)acrylate, a bisphenol A di(meth)acrylate, a terpene, a carotene, a divinyl (hetero)aromatic compound and a diisopropenyl (hetero)aromatic compound.

In some embodiments, the one or more polyfunctional monomers are at a level of about 2 to about 50 wt %, or about 2 to about 10 wt %, or about 10 to about 20 wt %, or about 20 to about 30 wt %, or about 30 to about 40 wt %, or about 40 to about 50 wt % of the sulfur polymer. In other embodiments, the one or more monofunctional monomers are at a level up to about 5 wt %, or about 10 wt %, or about 15 wt % of the sulfur polymer.

In some embodiments, the sulfur polymer is a thermoplastic or a thermoset. The sulfur polymer may be used in preparing elastomers, resins, lubricants, coatings, antioxidants, cathode materials for electrochemical cells, and dental adhesives/restorations. For example, the sulfur polymer may be formed into a polymeric film.

In certain embodiments, it can be desirable to use a nucleophilic viscosity modifier in liquefying the elemental sulfur or sulfur oligomers, for example, before adding the comonomers. For example, in certain embodiments, the elemental sulfur or sulfur oligomer is first heated with a viscosity modifier, then the viscosity-modified elemental sulfur or sulfur oligomer is heated with the comonomers. The nucleophilic viscosity modifier can be, for example, a phosphorus nucleophile (e.g., a phosphine), a sulfur nucleophile (e.g., a thiol) or an amine nucleophile (e.g., a primary or secondary amine). When the elemental sulfur or sulfur oligomer is heated in the absence of a nucleophilic viscosity modifier, the elemental sulfur or sulfur oligomer ring can open to form, e.g., diradicals, which can combine to form linear polymer chains which can provide a relatively high overall viscosity to the molten material. Nucleophilic viscosity modifiers can break these linear chains into shorter lengths, thereby making shorter polymers that lower the overall viscosity of the molten material, making the elemental sulfur or sulfur oligomer mixture easier to mix with and other species, and easier to stir for efficient processing. Some of the nucleophilic viscosity modifier will react to be retained as a covalently bound part of the polymer, and some will react to form separate molecular species, with the relative amounts depending on nucleophile identity and reaction conditions. While some of the nucleophilic viscosity modifier may end up as a separate molecular species from the polymer chain, as used herein, nucleophilic viscosity modifiers may become part of the polymer. Non-limiting examples of nucleophilic viscosity modifiers include triphenylphosphine, aniline, benzenethiol, and N,N-dimethylaminopyridine. Nucleophilic viscosity modifiers can be used, for example, in an amount up to about 10 wt %, or even up to about 5 wt % of the sulfur polymer. When a nucleophilic viscosity modifier is used, in certain embodiments it can be used in the range of about 5 wt % to about 15 wt % of the sulfur polymer.

In certain embodiments, a monofunctional comonomer can be used to reduce the viscosity of the sulfur polymer, for example, before adding other comonomers (e.g., before adding any polyfunctional comonomer). For example, in certain embodiments, the elemental sulfur or sulfur oligomer is first heated with one or more monofunctional comonomers. While not intending to be bound by theory, the inventors surmise that inclusion of monofunctional comonomers into the poly(sulfur) chains disrupts intermolecular associations of the elemental sulfur or sulfur oligomers, and thus decreases the viscosity. The monofunctional comonomer can be, for example, a mono(meth)acrylate such as benzyl methacrylate, a mono(oxirane) such as a styrene oxide or a glycidyl phenyl ether, or a mono(thiirane) such as t-butyl thiirane or phenoxymethylthiirane. A monofunctional comonomer can be used to modify the viscosity of the sulfur polymer, for example, in an amount up to about 10 wt %, up to about 5 wt %, or even up to about 2 wt % of the polymer. When a monofunctional monomer can be used to modify the viscosity of the sulfur polymer, in certain embodiments it can be used in the range of about 0.5 wt % to about 5 wt %, or even about 0.5 wt % to about 3 wt % of the sulfur polymer.

Of course, viscosity modification is not required, so in other embodiments the elemental sulfur or sulfur oligomers are heated together with the comonomers (and particularly with one or more polyfunctional comonomers) without viscosity modification. In other embodiments, a solvent, e.g., a halobenzene such as 1,2,4-trichlorobenzene, a benzyl ether, or a phenyl ether, can be used to modify the viscosity of the materials for ease of handling. The solvent can be added, for example, to the elemental sulfur or sulfur copolymers before reaction with a comonomer in order to reduce its viscosity, or to the polymerized material in order to aid in processing into a desired form factor.

The polymers described herein can be partially cured to provide a more easily processable material, which can be processed into a desired form (e.g., into a desired shape, such as in the form of a free-standing shape or a device), then fully cured in a later operation. For example, one embodiment of the invention is a method of making an article formed from the sulfur polymers as described herein. The method includes heating the sulfur polymer at a temperature in the range of about 120° C. to about 220° C. (e.g. 120° C. to about 150° C.) to form a prepolymer; forming the prepolymer into the shape of the article, to yield a formed prepolymer shape; and further heating the formed prepolymer shape to yield the article. The prepolymer can be formed, for example, by conversion of the one or more monomers at a level in the range of about 20 to about 50 mol %. For example, heating the sulfur polymer to form the prepolymer can be performed for a time in the range of about 20 seconds to about five minutes, for example, at a temperature in the range of about 175° C. to about 195° C. In one embodiment, the heating is performed for less than about 2 minutes at about 185° C. The person of skill in the art will determine the desired level of monomer conversion in the prepolymer stage to yield a processable prepolymer material, and will determine process conditions that can result in the desired level of monomer conversion.

In one embodiment, the prepolymer can be provided as a mixture with a solvent for forming, e.g., via casting, molding or printing. The prepolymers described herein can form miscible mixtures or solutions with a variety of nonpolar high-boiling aromatic solvents, including, for example, haloarene solvents such as di- and trichlorobenzene (e.g., 1,2,4-trichlorobenzene). The solvent can be added, for example, after the prepolymer is prepared, to provide a softened or flowable material suitable for a desired forming step (e.g., casting, molding, or spin-, dip- or spray-coating.) In some embodiments, the prepolymer/solvent mixture can be used at elevated temperatures (e.g., above about 100° C., above about 120° C. or above about 140° C.) to improve flow at relatively low solvent levels (e.g., for use in casting or molding processes). In other embodiments, the prepolymer/solvent mixture can be used at a lower temperature, for example, at ambient temperatures. The prepolymers described herein can remain soluble even after the solvent cools.

In one embodiment, the prepolymer is coated and cured as a film on a substrate. While S₈ is typically intractable due to its crystallinity, the materials described herein can be formed as to be amenable to solution processing (e.g., in molten or solvent-admixed form) to fabricate thin film materials. Mixtures of molten prepolymer and solvent can be diluted to the concentration desired for a given spin-coating process.

When forming thin films of the materials described herein on substrates, it can often be desirable to use a polyimide primer layer. Thus, a solution of a polyamic precursor (e.g., polypyromellitamic acid-4,4′-dianiline, or compounds with oxyaniline linkages), or similar polymer derivatives can be deposited onto a substrate and cured (e.g., by heating at a temperature in the range of about 120 to about 220° C.) to form a thin polyimide layer (e.g., as thin as 2 nm), upon which the materials described herein can be formed. Moreover, in many embodiments, even fully cured polymers as described herein can be melt-processed or suspended or dissolved in solvent and deposited on to substrates in a manner similar to those described for prepolymeric materials.

In certain embodiments, the prepolymer can be shaped and cured using a mold. For example, in one embodiment, the prepolymer (i.e., in liquid or solvent-admixed form) can be deposited (e.g., by pouring) into a TEFLON or silicone (e.g., polydimethylsiloxane (PDMS)) mold, then cured to form a desired shape. In another embodiment, a softened prepolymer material (e.g., swollen with solvent and/or softened by heat) can be imprinted by stamping with a mold bearing the desired inverse surface relief, then cured and allowed to cool. Moreover, in many embodiments, even fully cured copolymers as described herein can be shaped with a mold in a manner similar to those described for prepolymeric materials. Sulfur terpolymers and more complex polymer materials, such as in the form of cross-linked polymers, or non-crosslinked, intractable polymers, can be reprocessed by thermal or other stimuli activation of dynamic S—S bonds in the polymer system.

As described above, the sulfur polymers can be solution processed to fabricate articles. For example, another aspect of the invention is a method of forming an article formed from a sulfur polymer as described herein, the method comprising admixing the sulfur polymer with a nonpolar organic solvent (e.g., to make a suspension or solution), forming the admixed sulfur polymer into the shape of the article, and removing the solvent from the sulfur polymer to yield the article. The admixture with solvent can, for example, dissolve the sulfur polymer. Various process steps can be performed at elevated temperatures, for example, to decrease viscosity of the admixed sulfur polymer and to aid in evaporation of solvent.

For example, in one embodiment, a room temperature solution of any sulfur polymer described herein (e.g., in prepolymeric form) is poured into a TEFLON or PDMS mold. A decrease in viscosity at elevated temperatures (e.g., > about 140° C.) can allow sufficient flow into even intricate mold shapes. Once the mold is filled, it can be placed in a vacuum oven at increased temperature (e.g., about 210° C.) under ambient pressure to cure and to drive off solvent. For thicker molded samples, vacuum can be pulled on the solution when it is in a low viscosity state in order to ensure the removal of bubbles. The mold is then removed from the oven and allowed to cool before removal from the mold.

As the polymeric materials described herein can be effectively thermoplastic in nature, the person of skill in the art will understand that other methods familiar in the thermoplastic industries, such as injection molding, compression molding, and melt casting, can be used in forming articles from the materials described herein.

In still other embodiments, the present invention may feature an optical substrate comprising any of the sulfur polymers described herein. The optical substrate may be a substantially transparent optical body, such as a film, a lens, or a free-standing object. In some embodiments, the optical substrate has a refractive index in the range of about 1.7 to about 2.6 and at least one wavelength in the range of about 500 nm to about 10 μm. Preferably, the optical substrate has an optical transparency in the visible and infrared spectrum.

As used herein, the term “about” refers to plus or minus 10% of the referenced number.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

What is claimed is:

1. An iterative method of synthesizing a sulfur polymer, said method comprising:
a. providing elemental sulfur;
b. heating the elemental sulfur to form a molten sulfur comprising dynamic sulfur-sulfur (S—S) bonds, wherein heating enables thermal activation and scission of the dynamic S—S bonds from said molten sulfur to form sulfur radicals;
c. adding one or more first comonomers to the molten sulfur, wherein each first comonomer comprises at least one reactive functional group, wherein the one or more first comonomers and molten sulfur copolymerize by a reaction of the sulfur radicals with the reactive functional groups of the one or more first comonomers to form a sulfur prepolymer;
d. heating the sulfur prepolymer to form a molten sulfur prepolymer comprising the dynamic S—S bonds, wherein heating enables thermal activation and scission of the dynamic S—S bonds from said molten sulfur prepolymer to form sulfur-containing radical species; and
e. adding one or more second comonomers to the molten sulfur prepolymer, wherein each of the one or more second comonomers comprises at least one reactive functional group, wherein the one or more second comonomers and the molten sulfur prepolymer copolymerize by a reaction of the sulfur-containing radical species with the reactive functional groups of the one or more second comonomers to form the sulfur polymer;
wherein the iterative method improves processability of the elemental sulfur in producing the desired sulfur polymer by initially copolymerizing the elemental sulfur with the one or more first comonomers to prepare the sulfur prepolymer that is tailored to be miscibly or solubly compatible with the one or more second comonomers.

2. The method of claim 1, wherein the one or more first comonomers are selected to form the sulfur prepolymer having a tunable composition that enables miscibility or solubility of the sulfur prepolymer with the one or more second comonomers.

3. The method of claim 1, wherein the second comonomers are selected such that the second comonomers are soluble in the sulfur prepolymer, wherein the molten sulfur prepolymer is miscible with the one or more second comonomers.

4. The method of claim 1, wherein the second comonomers are different from the first comonomers.

5. The method of claim 1, wherein steps a.-e. are performed in sequence.

6. The method of claim 1, the one or more second comonomers are selected based on a number of reactive functional groups of each second comonomer, wherein the one or more second comonomers having an increasing number of reactive functional groups further enables branching or crosslinking in the sulfur polymer.

7. The method of claim 1, wherein the elemental sulfur is about 10-90 wt % of the sulfur polymer.

8. The method of claim 1, wherein the elemental sulfur is at least 50 wt % of the sulfur polymer.

9. The method of claim 1, wherein the first comonomers are about 5-50 wt % of the sulfur polymer.

10. The method of claim 1, wherein the one or more first comonomers are selected from a group consisting of amine comonomers, thiol comonomers, sulfide comonomers, alkynylly unsaturated comonomers, epoxide comonomers, nitrone comonomers, aldehyde comonomers, ketone comonomers, thiirane comonomers, ethylenically unsaturated comonomers, styrenic comonomers, vinylic comonomers, methacrylate comonomers, acrylonitrile comonomers, allylic monomers, acrylate monomers, vinylpyridine monomers, isobutylene monomers, maleimide monomers, norbornene monomers, monomers having at least one vinyl ether moiety, and monomers having at least one isopropenyl moiety

11. The method of claim 1, wherein the second comonomers are about 5-50 wt % of the sulfur polymer,

12. The method of claim 1, wherein the second comonomers are selected from a group consisting of amine comonomers, thiol comonomers, sulfide comonomers, alkynylly unsaturated comonomers, epoxide comonomers, nitrone comonomers, aldehyde comonomers, ketone comonomers, thiirane comonomers, ethylenically unsaturated comonomers, styrenic comonomers, vinylic comonomers, methacrylate comonomers, acrylonitrile comonomers, allylic monomers, acrylate monomers, vinylpyridine monomers, isobutylene monomers, maleimide monomers, norbornene monomers, monomers having at least one vinyl ether moiety, and monomers having at least one isopropenyl moiety.

13. The method of claim 1, wherein the elemental sulfur is melted at a temperature of about 120° C.-140° C.

14. The method of claim 1, further comprising adding a solvent to the sulfur prepolymer prior to heating the sulfur prepolymer.

15. The method of claim 1, wherein the solvent is 1,2-dichlorobenzene.

16. The method of claim 1, wherein the sulfur prepolymer is heated to a temperature of about 140° C.-200° C.

17. An iterative method of synthesizing a sulfur polymer, said method comprising:
a. providing elemental sulfur;
b. heating the elemental sulfur to form a molten sulfur comprising dynamic sulfur-sulfur (S—S) bonds, wherein heating enables thermal activation and scission of the dynamic S—S bonds from said molten sulfur to form sulfur radicals;
c. adding one or more unsaturated comonomers to the molten sulfur, wherein each unsaturated comonomer comprises at least one alkenyl functional group, wherein the one or more unsaturated comonomers and molten sulfur copolymerize by a reaction of the sulfur radicals with the alkenyl functional groups to form a sulfur prepolymer;
d. heating the sulfur prepolymer to form a molten sulfur prepolymer comprising the dynamic S—S bonds, wherein heating enables thermal activation and scission of the dynamic S—S bonds from said molten sulfur prepolymer to form sulfur-containing radical species; and
e. adding one or more second comonomers to the molten sulfur prepolymer, wherein each of the one or more second comonomers comprises at least one reactive functional group, wherein the one or more second comonomers and the molten sulfur prepolymer copolymerize by a reaction of the sulfur-containing radical species with the reactive functional groups of the one or more second comonomers to form the sulfur polymer;
wherein the iterative method improves processability of the elemental sulfur in producing the desired sulfur polymer by initially copolymerizing the elemental sulfur with the one or more unsaturated comonomers to prepare the sulfur prepolymer that is tailored to be miscibly or solubly compatible with the one or more second comonomers.

18. The method of claim 17, wherein the one or more unsaturated comonomers are selected to form the sulfur prepolymer having a tunable composition that enables miscibility or solubility of the sulfur prepolymer with the one or more second comonomers, and wherein the second comonomers are selected such that the second comonomers are soluble in the sulfur prepolymer.

19. The method of claim 17, wherein copolymerization occurs via a thiol-ene reaction.

20. An iterative method of synthesizing a sulfur polymer, said method comprising:
a. providing elemental sulfur;
b. heating the elemental sulfur to form a molten sulfur comprising dynamic sulfur-sulfur (S—S) bonds, wherein heating enables thermal activation and scission of the dynamic S—S bonds from said molten sulfur to form sulfur radicals;
c. adding one or more first comonomers to the molten sulfur, wherein each first comonomer comprises at least one reactive functional group, wherein the one or more first comonomers and molten sulfur copolymerize by a reaction of the sulfur radicals with the reactive functional groups of the one or more first comonomers to form a sulfur prepolymer;
d. dissolving the sulfur prepolymer in an organic solvent; and
e. adding one or more second comonomers to the dissolved sulfur prepolymer, wherein each of the one or more second comonomers comprises at least one reactive functional group, wherein the sulfur prepolymer is miscible with the one or more second comonomers, wherein the one or more second comonomers and the sulfur prepolymer copolymerize to form the sulfur polymer;
wherein the one or more first comonomers are selected to form the sulfur prepolymer having a tunable composition that enables miscibility or solubility of the sulfur prepolymer with the one or more second comonomers, and wherein the second comonomers are selected such that the second comonomers are soluble in the sulfur prepolymer, wherein the second comonomers are different from the first comonomers,
wherein the iterative method improves processability of the elemental sulfur in producing the desired sulfur polymer by initially copolymerizing the elemental sulfur with the one or more first comonomers to prepare the sulfur prepolymer that is tailored to be miscibly or solubly compatible with the one or more second comonomers.
